(19) United States
(12) Patent Application Publication
(10) Pub. No.: US 2015/0249217 A1
(43) Pub. Date: Sep. 3, 2015

(54) TRIAZINE-CONTAINING COMPOUND AND ORGANIC ELECTROLUMINESCENT DEVICE INCLUDING THE SAME

(71) Applicants: SAMSUNG DISPLAY CO., LTD., Yongin-City (KR); NATIONAL UNIVERSITY CORPORATION YAMAGATA UNIVERSITY, Yamagata-shi (JP)

(72) Inventors: Xiulan JIN, Yokohama (JP); Junji KIDO, Yonezawa-shi (JP); Hisahiro SASABE, Yonezawa-shi (JP); Yuichiro WATANABE, Yonezawa-shi (JP)

(21) Appl. No.: 14/632,432

(22) Filed: Feb. 26, 2015

(30) Foreign Application Priority Data
Feb. 28, 2014 (JP) 2014-038951

(51) Int. Cl. H01L 51/00; C07D 401/14

(52) U.S. Cl. CPC H01L 51/0067; C07D 401/14; H01L 51/0072; H01L 51/5072

(57) ABSTRACT

A triazine-containing compound and an organic electroluminescent device including the triazine-containing compound, the compound being represented by the following Formula 1:

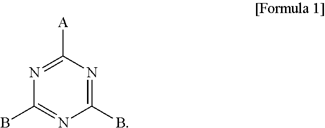

### CROSS-REFERENCE TO RELATED APPLICATION

Japanese Patent Application No. 2014-038951, filed on Feb. 28, 2014, in the Japanese Patent Office, and entitled: “Triazine Derivative and Organic Electroluminescent Device Using the Same,” is incorporated by reference herein in its entirety.

### BACKGROUND

1. Field

Embodiments relate to a triazine-containing compound and an organic electroluminescent device including the same.

2. Description of the Related Art

Triazine-containing compounds may be used in organic electroluminescent devices.

### SUMMARY

Embodiments are directed to a triazine-containing compound and an organic electroluminescent device including the same.

The embodiments may be realized by providing a triazine-containing compound represented by the following Formula 1:

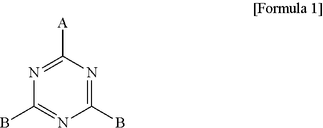

wherein, in Formula 1, A is an aryl group having 6 to 30 ring carbon atoms or a heteroaryl group having 5 to 30 ring carbon atoms, and each B is independently a phenylene group substituted with at least two azine rings.

A may be an aryl group having 6 to 30 ring carbon atoms.

Each B may independently be a phenylene group substituted with at least two pyridyl groups.

The phenylene group may be bound to the at least two pyridyl groups at position 3 or position 4 of the pyridyl groups.

The embodiments may be realized by providing an organic electroluminescent device including a triazine-containing compound, wherein the triazine-containing compound is represented by the following Formula 1:

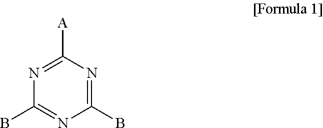

wherein, in Formula 1, A is an aryl group having 6 to 30 ring carbon atoms or a heteroaryl group having 5 to 30 ring carbon atoms, and each B is independently a phenylene group substituted with at least two azine rings.

A may be an aryl group having 6 to 30 ring carbon atoms.

Each B may independently be a phenylene group substituted with at least two pyridyl groups.

The phenylene group may be bound to the at least two pyridyl groups at position 3 or position 4 of the pyridyl groups.

The triazine-containing compound may be included in at least one of an electron transport layer and an emission layer.

### BRIEF DESCRIPTION OF THE DRAWINGS

Features will be apparent to those of skill in the art by describing in detail exemplary embodiments with reference to the attached drawings in which:

FIG. 1 illustrates a cross-sectional view of an organic electroluminescent device according to an embodiment;

FIG. 2 illustrates a ¹H-NMR spectrum of Precursor 5;

FIG. 3 illustrates a ¹H-NMR spectrum of Precursor 5 at a low magnetic field part;

FIG. 4 illustrates a mass spectrum of Precursor 5;

FIG. 5 illustrates a ¹H-NMR spectrum of B3PyPTZ according to an embodiment of the inventive concept;

FIG. 6 illustrates a ¹H-NMR spectrum of B3PyPTZ at a low magnetic field part;

FIG. 7 illustrates a mass spectrum of B3PyPTZ;

FIG. 8 illustrates a ¹H-NMR spectrum of B4PyPTZ according to an embodiment;

FIG. 9 illustrates a ¹H-NMR spectrum of B4PyPTZ at a low magnetic field part;

FIG. 10 illustrates a mass spectrum of B4PyPTZ;

FIG. 11 illustrates a graph of current density-voltage properties of B3PyPTZ and TPBi (Comparative Example);

FIG. 12 illustrates a graph of luminance-voltage properties of B3PyPTZ and TPBi;

FIG. 13 illustrates a graph of power efficiency-luminance properties of B3PyPTZ and TPBi;

FIG. 14 illustrates a graph of current efficiency-luminance properties of B3PyPTZ and TPBi;

FIG. 15 illustrates a graph of external quantum efficiency-luminance properties of B3PyPTZ and TPBi; and

FIG. 16 illustrates an EL spectrum of B3PyPTZ and TPBi.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. Like reference numerals refer to like elements throughout.

<1. Configuration of Triazine-Containing Compound>

The embodiments may provide a material that may decrease the driving voltage of an organic electroluminescent device, e.g., a triazine-containing compound (or triazine derivative). The triazine-containing compound may help decrease the driving voltage of the organic electroluminescent device particularly when used as an electron transport material and/or a host material of an emission layer. Here, the configuration of the triazine-containing compound according to an embodiment will be explained first. The triazine-containing compound according to an embodiment may be represented by the following Formula 1.

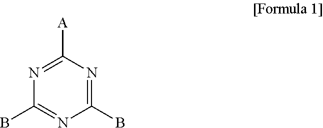

In the above Formula 1, A may be or may include, e.g., an aryl group having 6 to 30 ring carbon atoms or a heteroaryl group having 5 to 30 ring carbon atoms. In an implementation, A may be, e.g., an aryl group having 6 to 30 ring carbon atoms. Examples of the aryl group may include a phenyl group, a biphenyl group, a naphthyl group, an anthracenyl group, or the like. Examples of the heteroaryl group may include a furanyl group, a thienyl group, a benzofuranyl group, a benzothiophenyl group, a dibenzofuranyl group, a dibenzothiophenyl group, or the like, other than an azine ring group or moiety that will be described below. In an implementation, the aryl group and the heteroaryl group of A may be substituted with various suitable groups, e.g., functional groups.

B may be or may include, e.g., a phenylene group substituted with at least two azine rings. For example, the azine ring may be a heteroaromatic group or moiety that includes a nitrogen atom. Examples of the azine ring may include pyridine, pyrazine, pyrimidine, pyridazine, triazine, tetrazine, quinoline, isoquinoline, quinoxaline, quinazoline, cinnoline, or the like.

In an implementation, the azine ring may include pyridine. In an implementation, when the phenylene group is substituted with at least two pyridine groups (i.e., a pyridyl group), the phenylene may be bound to the pyridyl group at position 3 or position 4 of the pyridyl group. The azine ring may be substituted with suitable substituents. The phenylene group may also be substituted with a suitable substituent other than the azine ring.

As described in the following embodiments, the driving voltage of an organic electroluminescent device may be decreased by including the triazine-containing compound having the above-described configuration in at least one of an electron transport layer or an emission layer of the organic electroluminescent device. For example, electron injecting properties from a second electrode (e.g., cathode) may be improved by high electron accepting properties around the triazine moiety. In addition, a rigid network may be formed via a hydrogen bond between triazine-containing compounds. For example, a nitrogen atom in the azine ring may have an unshared electron pair, and the unshared electron pair may form the hydrogen bond with other hydrogen atoms in other triazine-containing compounds. Through the hydrogen bond, reinforced network between the triazine-containing compounds may be formed. The triazine-containing compounds may transports electron with high efficiency via the network. Thus, the driving voltage may be considered to be decreased.

In addition, driving voltage may be high when only one azine ring combined with or substituted on the phenylene group (see Comparative Examples described below). For example, the network between the triazine-containing compounds may become rigid when at least two azine rings are combined with the phenylene group. In addition, the network between the triazine-containing compounds may become particularly rigid when the phenylene group is bound to the pyridyl group at position 3 or position 4 of the pyridyl group.

Examples of the triazine-containing compound according to an embodiment may include B3PyPTZ, B4PyPTZ, B2PyPTZ, and B2QPyTZ, represented by the following Formulae 2 to 5.

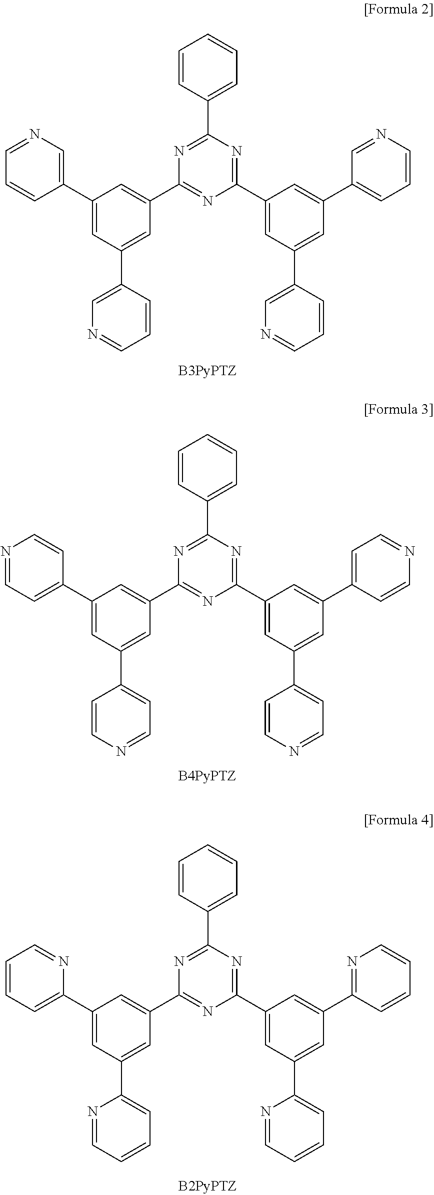 [SMILES: c1ccc(-c2nc(-c3cc(-c4cccnc4)cc(-c4cccnc4)c3)nc(-c3cc(-c4cccnc4)cc(-c4cccnc4)c3)n2)cc1 ; c1ccc(-c2nc(-c3cc(-c4ccncc4)cc(-c4ccncc4)c3)nc(-c3cc(-c4ccncc4)cc(-c4ccncc4)c3)n2)cc1 ; c1ccc(-c2nc(-c3cc(-c4ccccn4)cc(-c4ccccn4)c3)nc(-c3cc(-c4ccccn4)cc(-c4ccccn4)c3)n2)cc1]

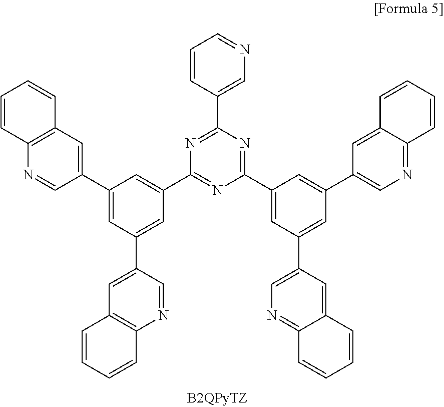 [SMILES: c1cncc(-c2nc(-c3cc(-c4cnc5ccccc5c4)cc(-c4cnc5ccccc5c4)c3)nc(-c3cc(-c4cnc5ccccc5c4)cc(-c4cnc5ccccc5c4)c3)n2)c1]

<2. Preparation Method of Triazine-Containing Compound>

Hereinafter, a method of preparing a triazine-containing compound will be explained. First, a reaction scheme for preparing B3PyPTZ and B4PyPTZ may be as follows.

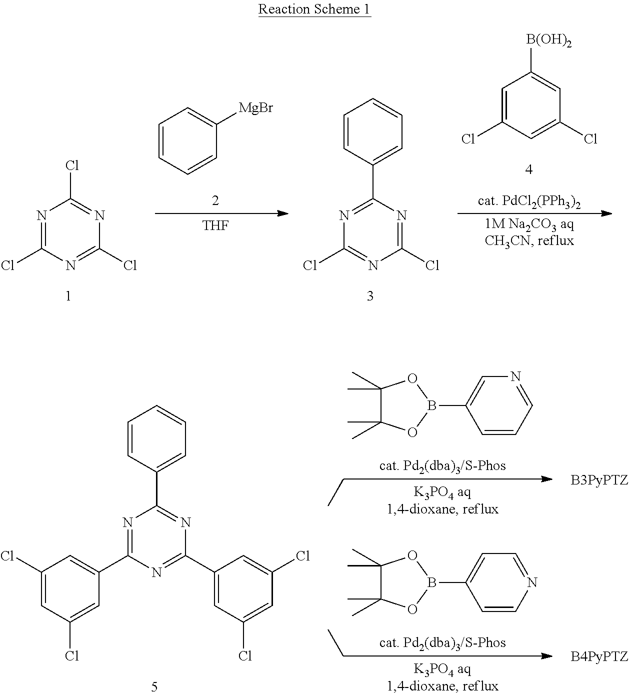

B3PyPTZ and B4PyPTZ may be prepared by the above-described reaction scheme (see the following synthetic examples for additional detail). In addition, by changing phenyl magnesium bromide of Precursor 2 into a desired aryl magnesium bromide or a heteroaryl magnesium bromide, a different Precursor 3 including a desired aryl group or heteroaryl group may be synthesized. In addition, by changing the boronic acid derivative of pyridine into a desired boronic acid derivative of an azine ring, a different triazine-containing compound including two desired azine rings in each phenylene group may be synthesized. For example, B2QPyTZ may be synthesized by the following reaction scheme.

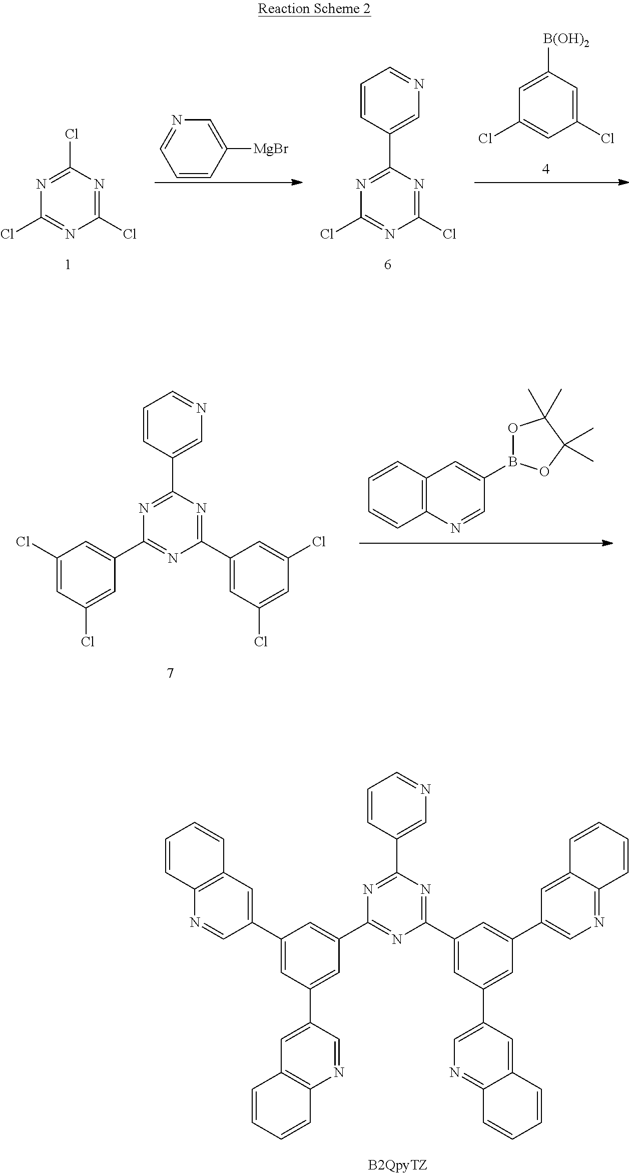

<3. Organic Electroluminescent Device Including Triazine-Containing Compound>

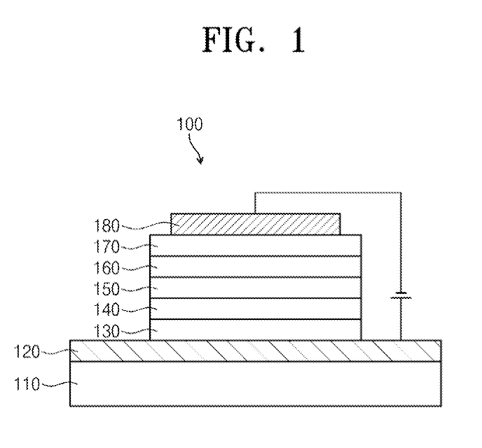

Then, an organic electroluminescent device including the triazine-containing compound according to an embodiment will be described in brief referring to FIG. 1. FIG. 1 illustrates a schematic cross-sectional view of an organic electroluminescent device according to an embodiment.

As shown in FIG. 1, an organic electroluminescent device 100 according to an embodiment may include a substrate 110, a first electrode 120 disposed on the substrate 110, a hole injection layer 130 disposed on the first electrode 120, a hole transport layer 140 disposed on the hole injection layer 130, an emission layer 150 disposed on the hole transport layer 140, an electron transport layer 160 disposed on the emission layer 150, an electron injection layer 170 disposed on the electron transport layer 160, and a second electrode 180 disposed on the electron injection layer 170.

Here, the triazine-containing compound according to an embodiment may be included in at least one of the electron transport layer 160 or the emission layer 150. In an implementation, the triazine-containing compound may be included in both the electron transport layer 160 and the emission layer 150.

Each organic thin film between the first electrode 120 and the second electrode 180 of the organic electroluminescent device may be formed by various suitable methods, e.g., a deposition method.

The substrate 101 may be a substrate used for a general organic electroluminescent device. For example, the substrate 110 may be a glass substrate, a semiconductor substrate, or a transparent plastic substrate.

The first electrode 120 may be, e.g., an anode, and may be formed on the substrate 110 by using a deposition method or a sputtering method. For example, the first electrode 120 may be formed using a metal having high work function, an alloy, a conductive compound, etc., as a transparent electrode. The first electrode 120 may be formed using, e.g., transparent and highly conductive indium tin oxide (ITO), indium zinc oxide (IZO), tin oxide (SnO₂), zinc oxide (ZnO), etc. In an implementation, the first electrode 120 may be formed as a reflection type electrode using magnesium (Mg), aluminum (Al), etc.

The hole injection layer 130 may be a layer for facilitating injection of holes from the first electrode 120 and may be formed, e.g., on the first electrode 120 to a thickness of from about 10 nm to about 150 nm. The hole injection layer 130 may be formed using suitable materials. The suitable materials may include, e.g., triphenylamine-containing polyether ketone (TPAPEK), 4-isopropyl-4′-methyldiphenyliodoniumtetrakis(pentafluorophenyl)borate (PPBI), N,N′-diphenyl-N,N′-bis-[4-(phenyl-m-tolyl-amino)-phenyl]-biphenyl-4,4′-diamine (DNTPD), a phthalocyanine compound such as copper phthalocyanine, 4,4′,4″-tris(3-methylphenylamino)triphenylamine (m-MTDATA), N,N′-di(1-natphtyl)-N,N′-diphenylbenzidine (NPB), 4,4′,4″-tris(N,N-diamino)triphenylamine (TDATA), 4,4′,4″-tris(N,N-2-naphthylamino)triphenyamine (2-TNATA), polyaniline/dodecylbenzenesulfonic acid (Pani/DBSA), poly(3,4-ethylenedioxythiophene)/poly(4-styrenesulfonate) (PEDOT/PSS), polyaniline/camphorsulfonic acid (Pani/CSA), polyaniline/poly(4-styrenesulfonate) (PANI/PSS), etc.

The hole transport layer 140 may be a layer including a hole transport material having hole transporting function and may formed, e.g., on the hole injection layer 130 to a thickness of from about 10 nm to about 150 nm. The hole transport layer 140 may be formed using a suitable hole transport material. The suitable hole transport material may include, e.g., 1,1-bis[(di-4-tolylamino)phenyl]cyclohexane (TAPC), a carbazole derivative such as N-phenyl carbazole, polyvinyl carbazole, etc., N,N′-bis(3-methylphenyl)-N,N′-diphenyl-[1,1-biphenyl]-4,4′-diamine (TPD), 4,4′,4″-tris(N-carbazolyl)triphenylamine (TCTA), N,N′-di(1-naphthyl)-N,N′-diphenylbenzidine (NPB), etc.

The emission layer 150 may be a layer emitting light via, e.g., fluorescence or phosphorescence. The emission layer 150 may be formed by including a host material and/or a dopant material as a light emitting material. In an implementation, the emission layer 150 may be formed to a thickness from about 10 nm to about 60 nm.

In an implementation, the triazine-containing compound according to an embodiment may be included as the host material of the emission layer 150. In an implementation, when the triazine-containing compound is included in the electron transport layer 160, it may not be necessary for the host material to be the triazine-containing compound. For example, a suitable host material may be included in the emission layer 150.

The suitable host material included in the emission layer 150 may include, e.g., tris(8-quinolinato)aluminum (Alq3), 4,4′-N,N′-dicarbazole-biphenyl (CBP), poly(n-vinylcarbazole) (PVK), 9,10-di(naphthalene-2-yl)anthracene (ADN), 4,4′,4″-tris(N-carbazolyl)triphenylamine (TCTA), 1,3,5-tris(N-phenybenzimidazole-2-yl)benzene (TPBI), 3-tert-butyl-9,10-di(natphto-2-yl)anthracene (TBADN), distyrylarylene (DSA), 4,4′-bis(9-carbazole)-2,2′-dimethyl-biphenyl (dmCBP), etc.

The emission layer 150 may be formed as an emission layer for emitting a specific color. For example, the emission layer 150 may be formed as a red emission layer, a green emission layer, and/or a blue emission layer.

When the emission layer 150 is the blue emission layer, suitable materials may be used as a blue dopant including, e.g., perylene or a derivative thereof, an iridium (Ir) complex such as bis[2-(4,6-difluorophenyl)pyridinate]picolinateiridium(III) (FIrpic), etc.

When the emission layer 150 is the red emission layer, suitable materials may be used as a red dopant including, e.g., rubrene or a derivative thereof, 4-dicyanomethylene-2-(p-dimethylaminostyryl)-6-methyl-4H-pyrane (DCM) or a derivative thereof, an iridium complex such as bis(1-phenylisoquinoline)(acetylacetonate)iridium(III) (Ir(piq)₂(acac), etc., an osmium (Os) complex, a platinum complex, etc.

When the emission layer 150 is the green emission layer, suitable materials may be used as a green dopant including, e.g., coumarin or a derivative thereof, an iridium complex such as tris(2-phenylpyridine)iridium(III) (Ir(ppy)₃), etc.

The electron transport layer 160 may be a layer including an electron transport material for transporting electrons and may be formed, e.g., on the emission layer 150 to a thickness from about 15 nm to about 50 nm. The triazine-containing compound according to an embodiment may be used as the electron transport material. In an implementation, in the case that the triazine-containing compound is included in the emission layer, e.g., the triazine-containing compound is used as the host material of the emission layer, it may not be necessary for the electron transport material to be or include the triazine-containing compound according to this embodiment. For example, the electron transport layer 160 may be formed using suitable electron transport materials. The suitable electron transport material may include, e.g., a quinoline derivative such as Alq3, a 1,2,4-triazole derivative (TAZ), bis(2-methyl-8-quinolinolato)-(p-phenylphenolate)-aluminum (BAlq), berylliumbis(benzoquinoline-10-olate (BeBq2), a Li complex such as lithium quinolate (LIQ), etc.

The electron injection layer 170 may be a layer for facilitating injection of electrons from the second electrode 180 and may be formed to a thickness from about 0.3 nm to about 9 nm. In an implementation, the electron injection layer 170 may be formed using suitable materials, e.g., may be formed using lithium fluoride (LiF), sodium chloride (NaCl), cesium fluoride (CsF), lithium oxide (Li₂O), barium oxide (BaO), etc.

The second electrode 180 may be, e.g., a cathode. For example, the second electrode 180 may be formed as a reflection type electrode using a metal having small work function, an alloy, a conductive compound, etc. The second electrode 180 may be formed using, e.g., lithium (Li), magnesium (Mg), aluminum (Al), aluminum-lithium (Al—Li), calcium (Ca), magnesium-indium (Mg—In), magnesium-silver (Mg—Ag), etc. In addition, the second electrode 180 may be formed as a transparent electrode using ITO, IZO, etc. The second electrode 180 may be formed on the electron injection layer 170 by using a deposition method or a sputtering method.

As described above, the structure of the organic electroluminescent device 100 according to this embodiment were explained. In the organic electroluminescent device 100 including the triazine-containing compound according to this embodiment, a rigid network may be formed between the triazine-containing compounds, and electron transport properties may be improved and the driving voltage may be decreased.

In an implementation, the structure of the organic electroluminescent device 100 according to exemplary embodiments may not be limited to the above-described embodiments. The organic electroluminescent device 100 according to exemplary embodiments may be formed using the structures of various other suitable organic electroluminescent devices. For example, the organic electroluminescent device 100 may not include at least one of the hole injection layer 130, the hole transport layer 140, the electron transport layer 160 and the electron injection layer 170. In an implementation, each layer of the organic electroluminescent device 100 may be formed as a single layer or as a multilayer.

In an implementation, the organic electroluminescent device 100 may be further provided with a hole inhibiting layer between the hole transporting layer 140 and the emission layer 150 to prevent the diffusion of triplet excitons or holes to the electron transport layer 160. In an implementation, the hole inhibiting layer may be formed using, e.g., an oxadiazole derivative, a triazole derivative, a phenanthroline derivative, etc.

### EXAMPLES

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, the organic electroluminescent device according to an embodiment will be described in particular with the Examples and Comparative Examples.

### Synthetic Example 1

### Synthesis of Precursor 3

Precursor 3 was synthesized according to a suitable method

### Synthetic Example 2

### Synthesis of Precursor 5

In a 300 mL four-necked flask equipped with a nitrogen inlet, a dimroth (condenser), and a mechanical stirrer, 11.5 g (50.9 mmol) of dichlorophenyl triazine, 21.4 g (112 mmol) of dichlorophenylboronic acid, 600 mL of CH₃CN, and 200 mL of a 1 M Na₂CO₃ aqueous solution were added, followed by N₂ bubbling for 2 hours. Then, 1.79 g (2.55 mmol) of PdCl₂(PPh₃)₂ was added thereto, followed by heating and refluxing while stirring. After 20 hours, the disappearance of raw materials was checked, and the reactant was allowed to stand and cool. The reactant was transferred to a 2,000 mL Erlenmeyer flask, 500 mL of water was added thereto and stirred, and salt was removed. By means of suction filtering using a glass filter, filtrate was separated and purified by column chromatography to produce a target material (yield 8.2 g, yield 57%).

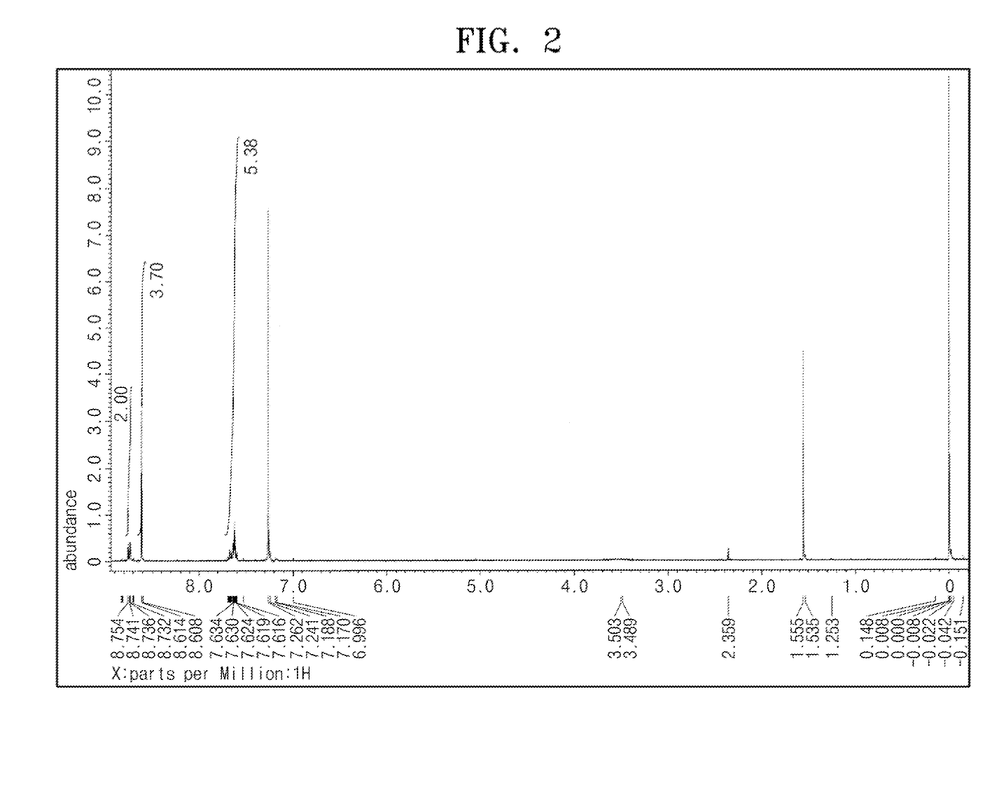

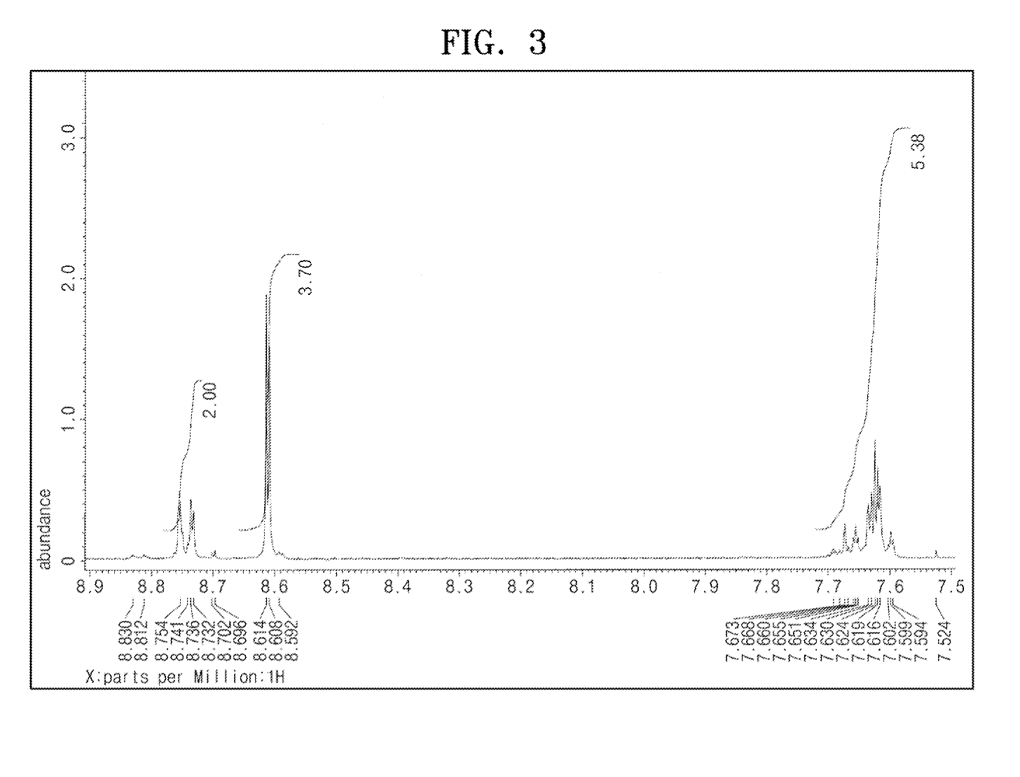

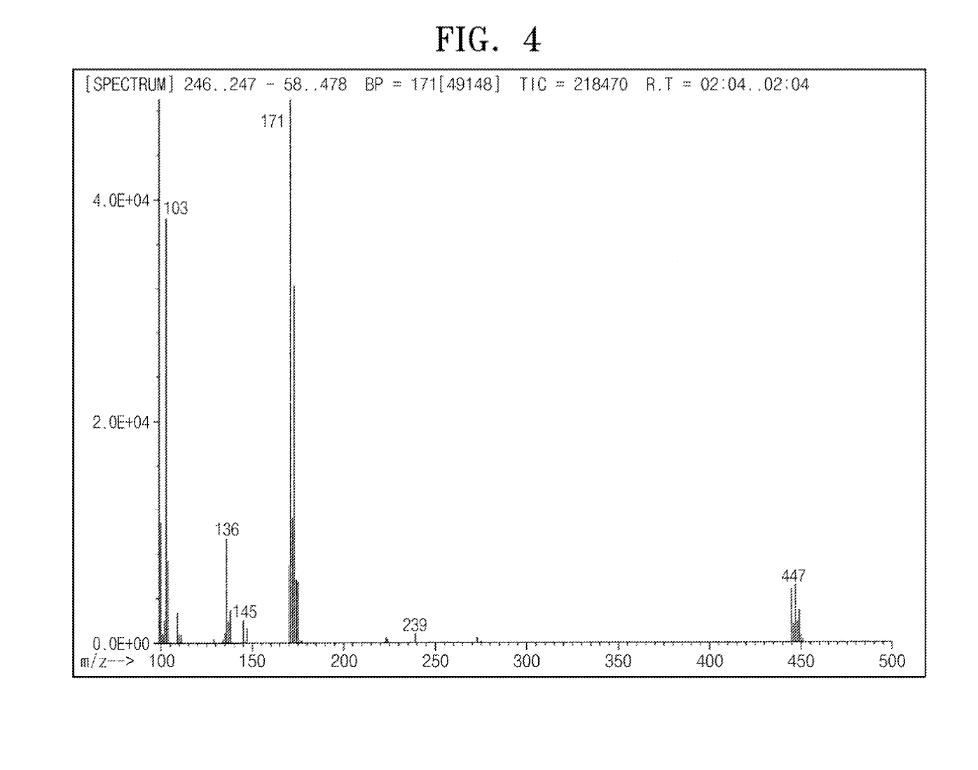

In addition, ¹H-NMR (400 MHz, CDCl₃) of the target material was measured and the following chemical shifts were obtained (unit ppm, the same hereinafter). 8.73-8.70 (m, 2H), 8.58 (d, J=2.0 Hz, 4H), 7.69-7.52 (m, 5H). FIGS. 2 and 3 illustrate NMR spectra. FIG. 3 illustrates a spectrum at a low magnetic field part in FIG. 2. In addition, the mass spectrum of the target material was measured and m/z=447[M]⁺ was obtained. The mass spectrum is shown in FIG. 4. From the results, the target material was determined to be Precursor 5.

### Synthetic Example 3

### Synthesis of B3PyPTZ

In a 200 mL three-necked flask equipped with a nitrogen inlet, a dimroth (condenser), and a magnetic stirrer, 1.14 g (2.55 mmol) of Precursor 5, 2.63 g (12.8 mmol) of 3-pyridineboronic acid ester, 40 mL of 1,4-dioxane, and 13 mL of a 1.35 M K₃PO₄ aqueous solution were added, followed by N₂ bubbling for 3 hours. Then, 0.048 g (0.052 mmol) of Pd₂(dba)₃ and 0.044 g (0.107 mmol) of 2-dicyclohexylphosphino-2′,6′-dimethoxybiphenyl (S-Phos) were added thereto, followed by heating and refluxing while stirring vigorously. After 32 hours, the disappearance of raw materials was confirmed by thin layer chromatography (TLC), and the reactant was allowed to stand and cool. By means of suction filtering, filtrate was separated, and salt was removed from the filtrate by using water. The filtrate was dissolved, and a target material was obtained by column chromatography (yield 1.42 g, 90%).

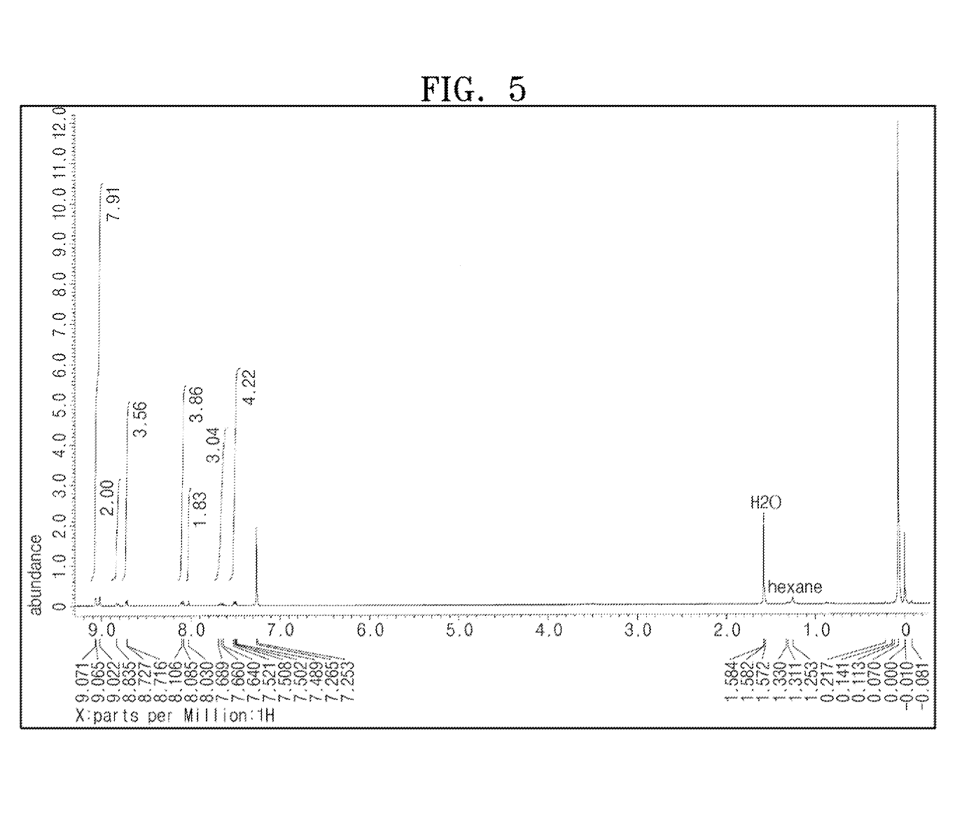

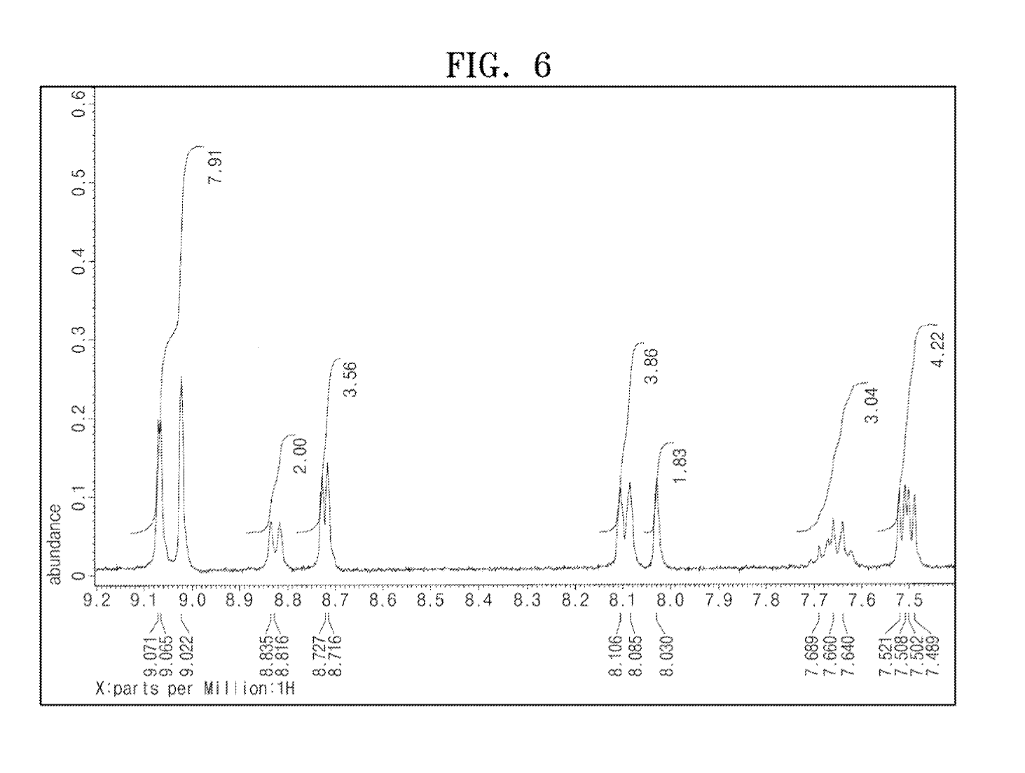

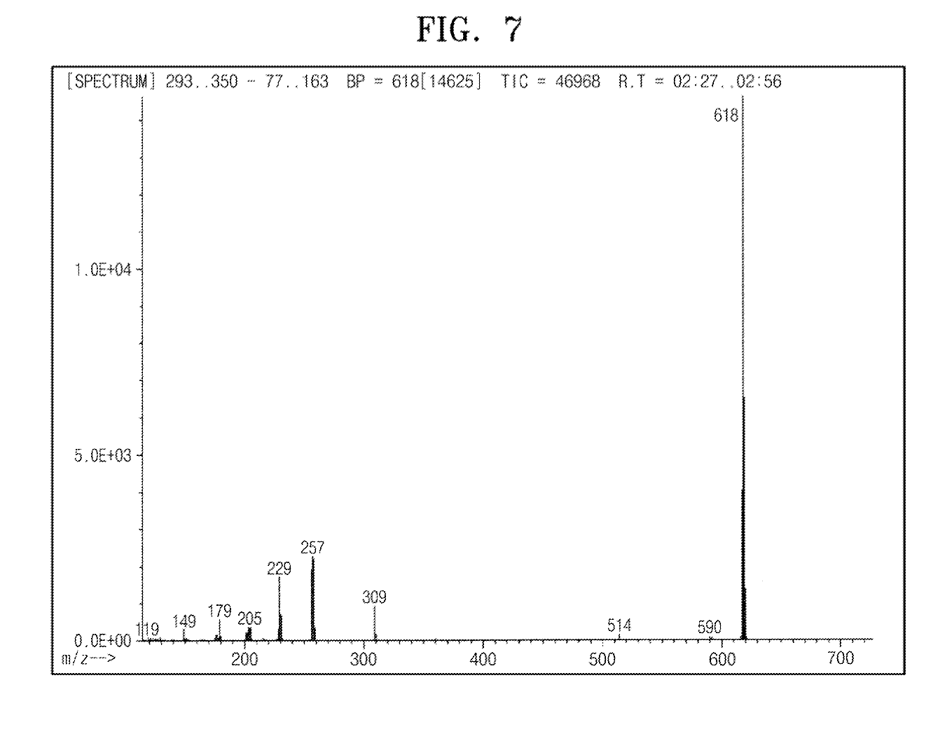

¹H-NMR (400 MHz, CDCl₃) of the target material was measured and the following chemical shifts were obtained. 9.07 (d, 4H, J=2.4 Hz), 9.02 (s, 4H), 8.83 (d, 2H, J=7.6 Hz), 8.72 (d, 4H, J=4.4 Hz), 8.10 (d, 4H, J=8.4 Hz), 8.03 (s, 2H), 7.69-7.64 (m, 3H), 7.51 (dd, 4H, J=5.2, 5.2 Hz). FIGS. 5 and 6 illustrate NMR spectra. FIG. 6 illustrates a spectrum at a low magnetic field part in FIG. 5. In addition, the mass spectrum of the target material was measured and m/z=618 [M]⁺ (Anal. Calcd for C₄₁H₂₈N₇: C, 79.72; H, 4.41; N, 15.87%. Found: C, 79.52; H, 4.25; N, 15.90%.) was obtained. The mass spectrum was illustrated in FIG. 7. From the results, the target material was determined to be B3PyPTZ.

### Synthetic Example 4

### Synthesis of B4PyPTZ

In a 100 mL three-necked flask equipped with a nitrogen inlet, a dimroth (condenser), and a magnetic stirrer, 1.20 g (2.68 mmol) of Precursor 5, 2.75 g (13.4 mmol) of 4-pyridineboronic acid ester, 40 mL of 1,4-dioxane, and a 1.35 M K₃PO₄ aqueous solution (3.82 g of K₃PO₄ dissolved in 13.3 mL of H₂O) were added, followed by N₂ bubbling for 1.5 hours. Then, 0.050 g (0.055 mmol) of Pd₂(dba)₃ and 0.046 g (0.112 mmol) of 2-dicyclohexylphosphino-2′,6′-dimethoxybiphenyl (S-Phos) were added thereto, followed by heating and refluxing while stirring vigorously. After 43 hours, the disappearance of raw materials was confirmed by TLC, 50 mL of water was added to dissolve salt, followed by stirring, and the reactant was allowed to stand and cool. Precipitated solid was recovered, and salt was removed from filtrate by using water. Through column chromatography, a target material was obtained (yield 1.40 g, 85%).

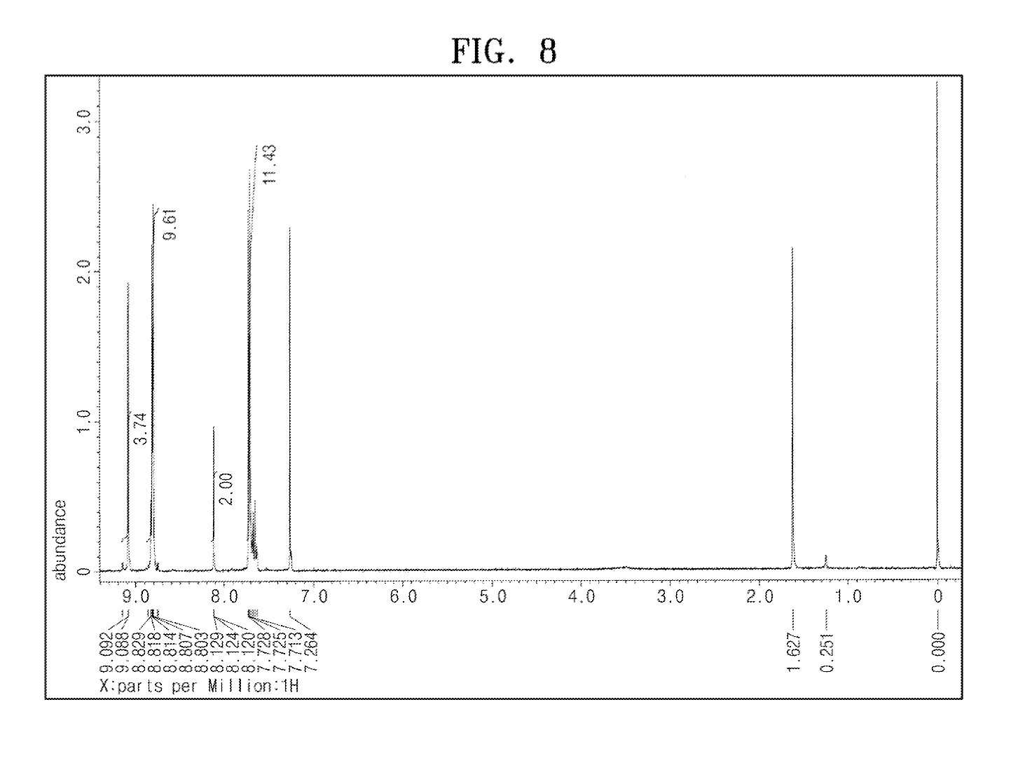

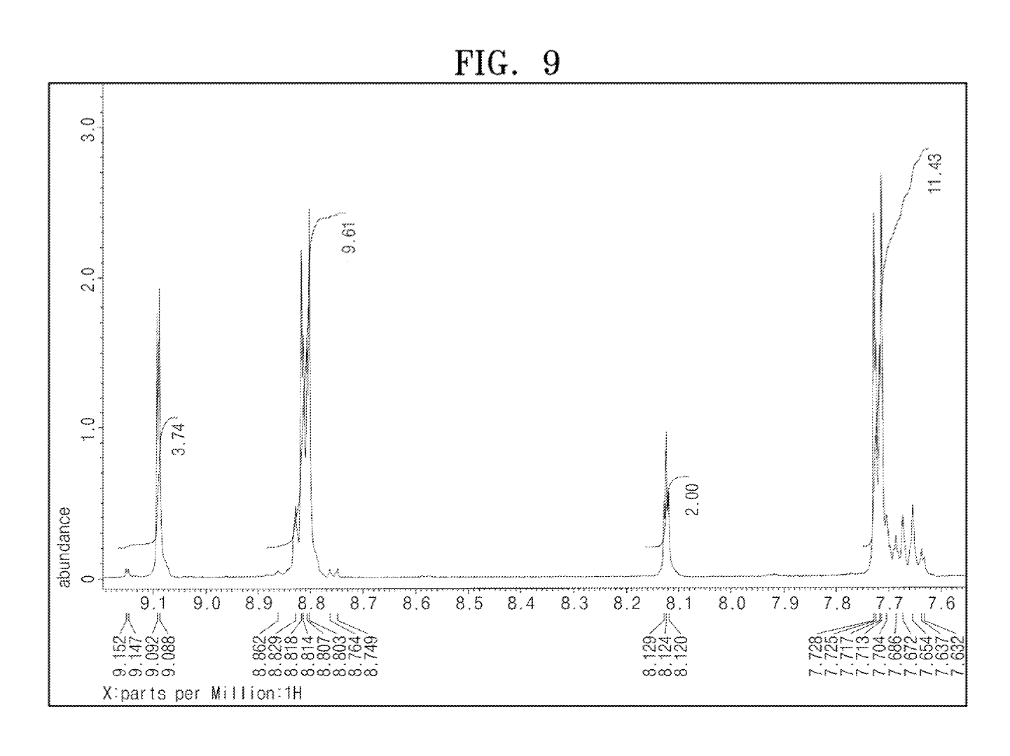

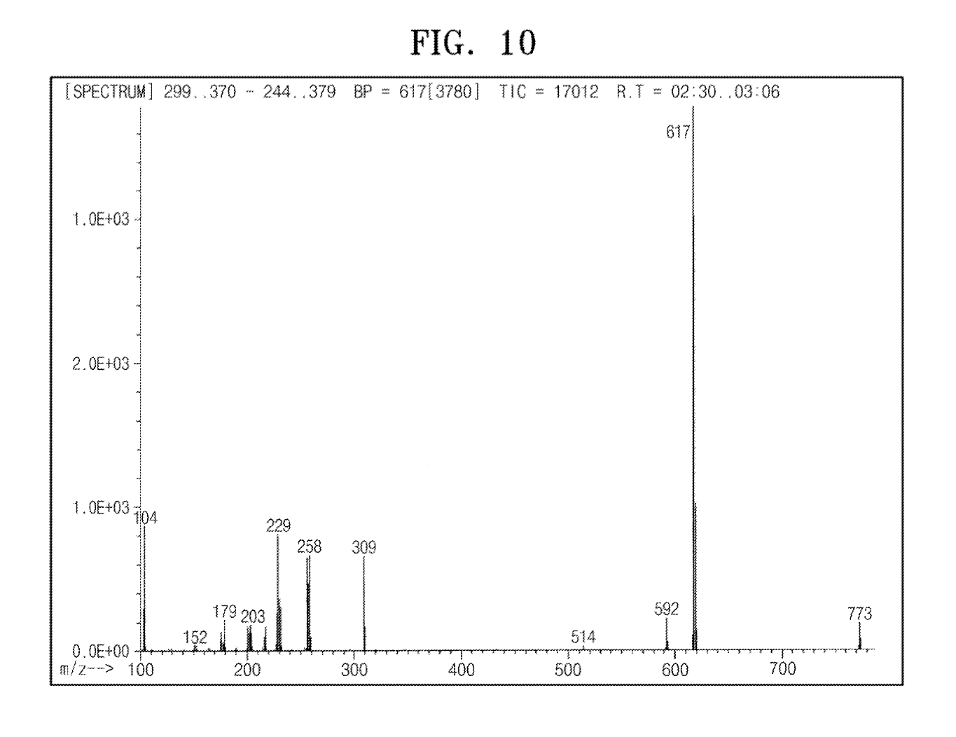

¹H-NMR (400 MHz, CDCl₃) of the target material was measured and the following chemical shifts were obtained. 9.09 (d, 4H, J=1.6 Hz), 8.86-8.75 (m, 10H), 8.13 (d, 2H, J=3.6 Hz), 7.73-7.63 (m, 11H). FIGS. 8 and 9 illustrate NMR spectra. FIG. 9 illustrates a spectrum at a low magnetic field part in FIG. 8. In addition, the mass spectrum of the target material was measured and m/z=617[M]⁺ (Anal. Calcd for C₄₁H₂₈N₇: C, 79.72; H, 4.41; N, 15.87%. Found: C, 79.81; H, 4.36; N, 15.97%.) was obtained. The mass spectrum was illustrated in FIG. 10. From the results, the target material was determined to be B4PyPTZ.

### Synthetic Example 5

### Synthesis of B2PyPTZ

A target material was obtained by performing the same procedure described in Synthetic Example 3, except for using 2.63 g of 2-pyridineboronic acid ester instead of 3-pyridineboronic acid ester (yield 1.40 g, 89%).

The mass spectrum of the target material was measured and m/z=618[M]⁺ (Anal. Calcd for C₄₁H₂₈N₇: C, 79.72; H, 4.41; N, 15.87%. Found: C, 79.52%; H, 4.25%; N, 15.90%.) was obtained. From the results, the target material was determined to be B2PyPTZ.

### Synthetic Example 6

### Synthesis of B2QPyPZ

Precursor 6 was synthesized by performing the same procedure described in Synthetic Example 1 except for using 3-pyridine magnesium bromide instead of phenyl magnesium bromide.

Precursor 7 was obtained by performing the same procedure described in Synthetic Example 2 except for using 11.6 g of Precursor 6 instead of dichlorophenyltriazine (yield 7.10 g, 31%). Then, B2QPyTZ was obtained by performing the same procedure described in Synthetic Example 3 except for using 1.14 g of Precursor 7 instead of Precursor 5 and using 3.26 g of 3-quinolineboronic acid ester instead of 3-pyridineboronic acid ester (yield 1.71 g, 82%).

The mass spectrum of the target material was measured and m/z=819[M]⁺ (Anal. Calcd for C₅₆H₃₄N₈: C, 82.12; H, 4.19; N, 13.69%. Found: C, 82.12; H, 4.19; N, 13.69%.) was obtained.

(Manufacture of Organic Electroluminescent Device)

Then, an organic electroluminescent device was manufactured by the following method. First, with respect to an ITO-glass substrate patterned and washed in advance, surface treatment was performed using ozone (O₃). The layer thickness of an ITO layer (first electrode) was about 130 nm. After the ozone treatment, the substrate was washed. The washed substrate was set on a glass bell jar type evaporator for forming an organic layer, and a hole injection layer, a hole transport layer, an emission layer, and an electron transport layer were deposited one by one under the vacuum degree of 10⁻⁴ to 10⁻⁵ Pa. Subsequently, the substrate was transferred to a glass bell jar type evaporator for forming a metal layer, and an electron injection layer and a cathode material were deposited one by one under the vacuum degree of 10⁻⁴ to 10⁻⁵ Pa.

Here, TPAPEK and PPBI were used as hole injection materials. Specifically, the hole injection layer was formed by co-depositing the materials. The thickness of the hole injection layer was about 20 nm. TAPC was used as a hole transport material. The thickness of the hole transport layer was about 30 nm. The host of a light emitting material was CBP (Examples 1 to 4, Comparative Examples 1 to 3) or B3PyPTZ (Example 5). Dopant was Ir(ppy)₃. The amount doped of the dopant was about 8 wt % with respect to the amount of the host. Specifically, by co-depositing the materials on the hole transport layer, the emission layer was formed. The thickness of the emission layer was about 10 nm. As the electron transport material, B3PyPTZ (Examples 1 and 5), B4PyPTZ (Example 2), B2PyPTZ (Example 3), B2QPyTZ (Example 4), TPBi (Comparative Example 1), ETM 1 (Comparative Example 2) or ETM 2 (Comparative Example 3) were used. The thickness of the electron transport layer was about 50 nm. The structures of ETM 1 and ETM 2 are illustrated in the following Formulae 8 and 9.

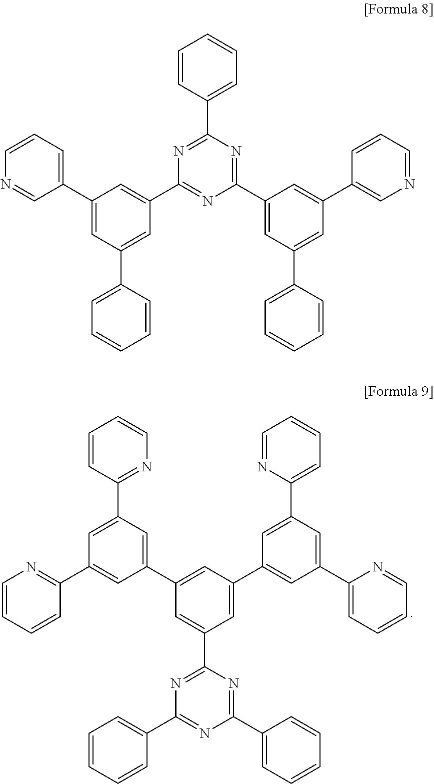 [SMILES: c1ccc(-c2cc(-c3cccnc3)cc(-c3nc(-c4ccccc4)nc(-c4cc(-c5ccccc5)cc(-c5cccnc5)c4)n3)c2)cc1 ; c1ccc(-c2nc(-c3ccccc3)nc(-c3cc(-c4cc(-c5ccccn5)cc(-c5ccccn5)c4)cc(-c4cc(-c5ccccn5)cc(-c5ccccn5)c4)c3)n2)cc1]

In addition, ETM 1 and ETM 2 were synthesized by a suitable method and by changing each material in the above-described reaction scheme. LiF was used as the electron injection material. The thickness of the electron injection layer was about 0.5 nm. Al was used as the material of the second electrode. The thickness of the second electrode was about 100 nm.

The formation of a layer of an organic compound was conducted by a resistance heating type deposition method at a depositing rate of about 0.1-5.0 Å/sec. The deposition of LiF was performed by the same deposition method at a depositing rate of about 0.01-0.1 Å/sec. The layer formation of Al was performed by the same deposition method at a depositing rate of about 5.0-20.0 Å/sec. In addition, the control of a layer thickness was performed by using a quartz oscillator type layer-forming controller. According to the above-described procedure, an organic electroluminescent device (a green phosphorescent device) was manufactured.

(Measuring Luminance)

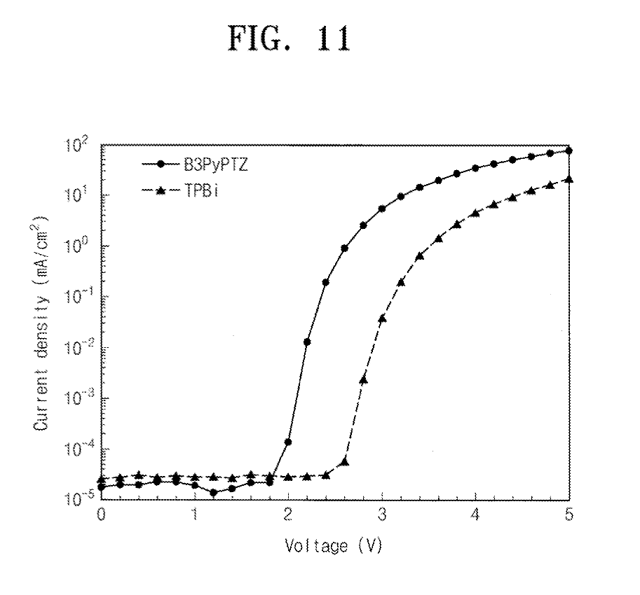

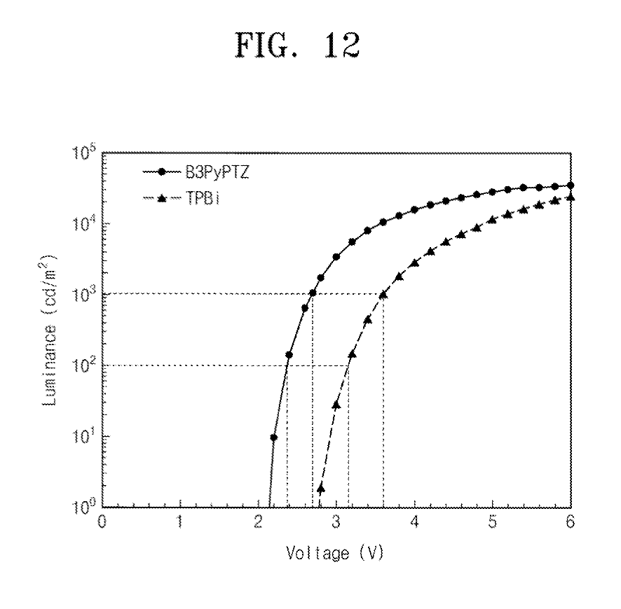

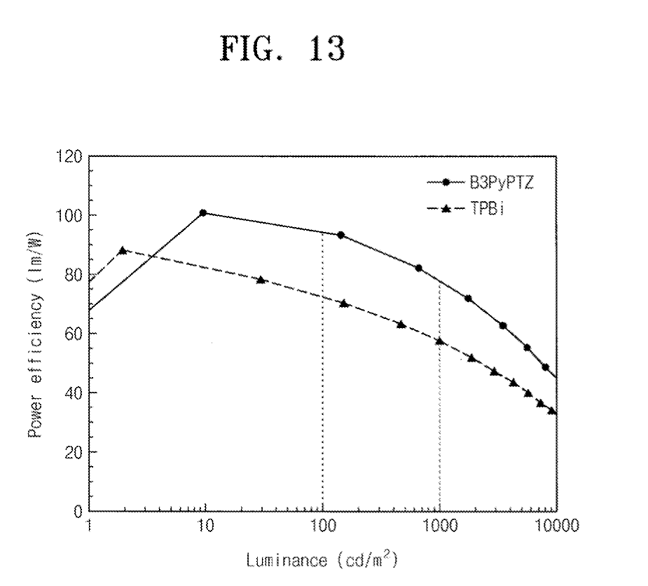

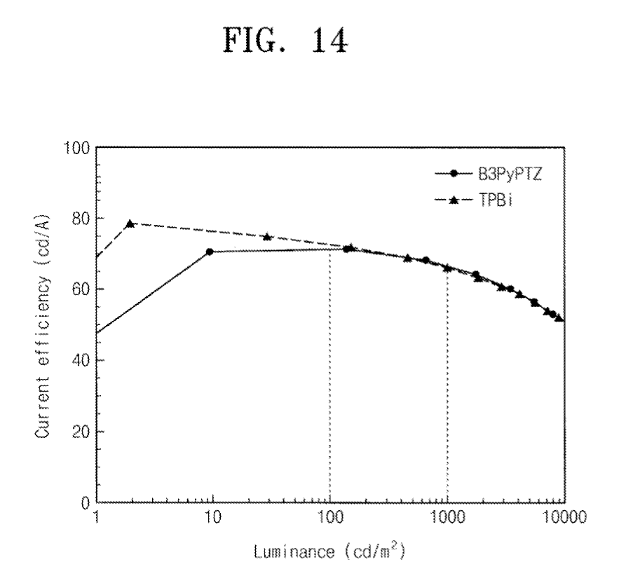

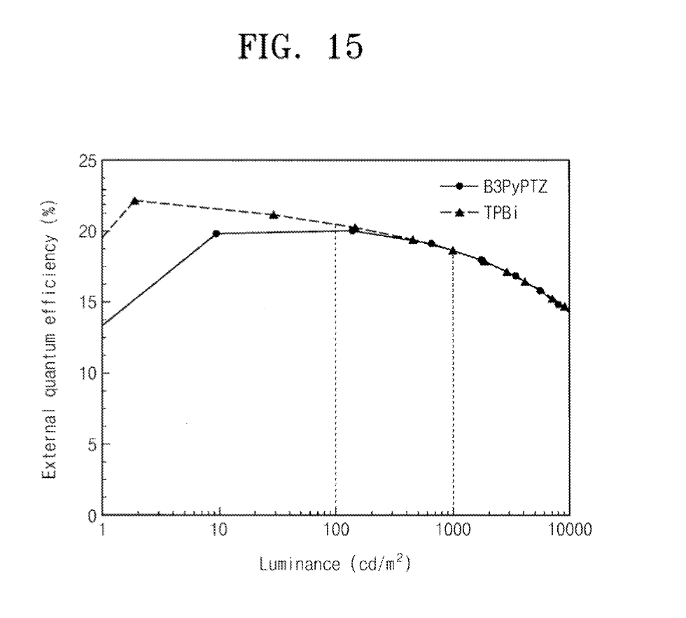

Luminance was measured by using a source meter of 2400 series manufactured by Keithley Instruments Co., a chroma meter CS-200 (manufactured by Konica Minolta Holdings Co., Ltd.), a measuring angle of 1°), and a PC program for measuring of LabVIEW 8.2 (produced by Japanese National Instruments Co., Ltd.) in a dark room. Measuring conditions were: [a voltage set mode, a DC mode], a voltage step width of 0.2 V, and a light emission area of 4.0 mm². Based on the measured results, current density-voltage properties, luminance-voltage properties, power efficiency-luminance properties, current efficiency-luminance properties and external quantum efficiency-luminance properties were evaluated. The results are illustrated in FIGS. 11 to 15 and Table 1. In addition, the properties of B2PyPTZ, B2QPyTZ and B4PyPTZ were similar to those of B3PyPTZ, and the properties of B2PyPTZ, B2QPyTZ and B4PyPTZ are not shown in FIGS. 11 to 15. In addition, even though the properties of ETM 1 and 2 are not shown in FIGS. 11 to 15, similar properties were obtained as those of TPBi.

TABLE 1

| | | Electron transport | Voltage (V) |
|---|---|---|---|
| | Host | material | @100 cd/m² |
| Example 1 | CBP | B3PyPTZ | 2.3 |
| Example 2 | CBP | B4PyPTZ | 2.3 |
| Example 3 | CBP | B2PyPTZ | 2.6 |
| Example 4 | CBP | B2PyPTZ | 2.4 |
| Example 5 | B3PyPTZ | B3PyPTZ | 2.4 |
| Comparative Example 1 | CBP | TPBi | 3.1 |
| Comparative Example 2 | CBP | ETM1 | 2.8 |
| Comparative Example 3 | CBP | ETM2 | 2.9 |

(Measuring Electroluminescent (EL) Spectrum)

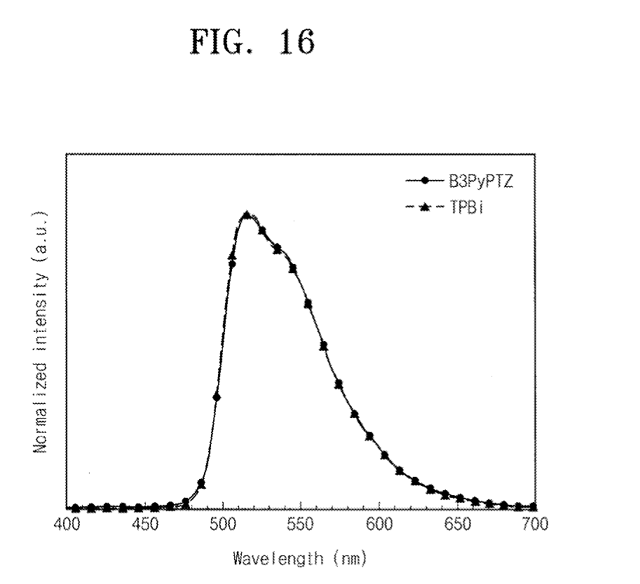

EL spectrum was measured by using a photo multi channel analyzer, PMA-11 (manufactured by Hamamatsu photonics Co., Ltd.), which is a spectrophotometric apparatus including a spectrometer and a multi channel detecting device in a body, and a source meter of 2400 series manufactured by Keithley Instruments Co. Basic software of U6039-01version 8.2 (produced by Hamamatsu photonics Co., Ltd.) for PMA was used as a PC program for measuring, and measuring conditions include an optional time period (about 19 ms˜) of the exposing time of a detector, the wavelength from about 299.6 to about 800.4 nm, and an optional value (mA) of a current value. The results are shown in FIG. 16. In addition, since the spectra of B2PyPTZ, B2QPyTZ and B4PyPTZ were similar to that of B3PyPTZ, the spectra of B2PyPTZ, B2QPyTZ and B4PyPTZ are not shown in FIG. 16.

High external quantum efficiency and extremely low driving voltage were realized for an organic electroluminescent device using B3PyPTZ, even though the device had a common device structure. Particularly, at 100 cdm⁻², a driving voltage of about 2.3 V, an external quantum efficiency of about 20%, a current efficiency of about 71 cdA⁻¹, and a power efficiency of about 961 mW⁻¹ were exhibited. When compared to an organic electroluminescent device (Comparative Example 1) having the same structure and using TPBi as an electron transport material, the external quantum efficiency was the same degree, however markedly decreased effects of the driving voltage by about 0.7 V were obtained. In addition, markedly decreased effects of the driving voltage by about 0.5 to 0.6 V were obtained when compared to those devices using ETM 1 and 2. An organic electroluminescent device using a triazine-containing compound according to another embodiment also illustrated similar properties as those of B3PyPTZ.

First, the improvement of electron injection properties from the second electrode (cathode) due to the high electron accepting properties around a triazine moiety may be considered for the reason. Second, the combination of a triazine-containing compound with another triazine-containing compound by two azine rings on a phenylene group via a hydrogen bond may be considered. Third, the combination of a triazine-containing compound with another triazine-containing compound by two azine rings on the phenylene group via a hydrogen bond may be considered. For example, a rigid network may be formed between the triazine-containing compounds via the hydrogen bond, and the network may contribute to the improvement of the electron transport properties. In addition, when comparing Examples 1 to 3, Examples 1 and 2 (in which the phenylene group was bound to the pyridyl group at position 3 or position 4 of the pyridyl group) exhibited lower driving voltages than Example 3 (in which the phenylene group was bound to the pyridyl group at position 2 of the pyridyl group). Thus, it may be seen that the network between the triazine-containing compounds in which the phenylene group is bound to the pyridyl group at position 3 or 4 of the pyridyl group may be particularly rigid.

By way of summation and review, a triazine-containing compound may be substituted with a same substituent at positions 2, 4, and 6 of the triazine moiety. In addition, a triazine-containing compound may include two of three phenyl groups combined at positions 2, 4, and 6 of the triazine moiety, which may each be substituted with one pyridyl group.

Some organic electroluminescent devices including a triazine-containing compound may have a very high driving voltage and no practical use.

The embodiments may provide a triazine-containing compound that may help decrease the driving voltage of an organic electroluminescent device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A triazine-containing compound represented by the following Formula 1: 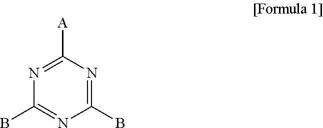
wherein, in Formula 1, A is an aryl group having 6 to 30 ring carbon atoms or a heteroaryl group having 5 to 30 ring carbon atoms, and each B is independently a phenylene group substituted with at least two azine rings.

2. The triazine-containing compound as claimed in claim 1, wherein A is an aryl group having 6 to 30 ring carbon atoms.

3. The triazine-containing compound as claimed in claim 1, wherein each B is independently a phenylene group substituted with at least two pyridyl groups.

4. The triazine-containing compound as claimed in claim 3, wherein the phenylene group is bound to the at least two pyridyl groups at position 3 or position 4 of the pyridyl groups.

5. An organic electroluminescent device comprising a triazine-containing compound, wherein the triazine-containing compound is represented by the following Formula 1: 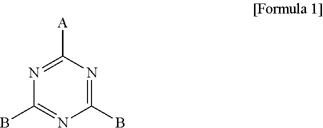
wherein, in Formula 1, A is an aryl group having 6 to 30 ring carbon atoms or a heteroaryl group having 5 to 30 ring carbon atoms, and each B is independently a phenylene group substituted with at least two azine rings.

6. The organic electroluminescent device as claimed in claim 5, wherein A is an aryl group having 6 to 30 ring carbon atoms.

7. The organic electroluminescent device as claimed in claim 5, wherein each B is independently a phenylene group substituted with at least two pyridyl groups.

8. The organic electroluminescent device as claimed in claim 7, wherein the phenylene group is bound to the at least two pyridyl groups at position 3 or position 4 of the pyridyl groups.

9. The organic electroluminescent device as claimed in claim 5, wherein the triazine-containing compound is included in at least one of an electron transport layer and an emission layer.
